(19) United States
(12) Patent Application Publication
(10) Pub. No.: US 2024/0130775 A1
(43) Pub. Date: Apr. 25, 2024

(54) CRYOTHERAPEUTIC SYSTEMS AND METHODS FOR TARGETED LUNG NEUROMODULATION THERAPIES

(71) Applicant: Ryme Medical, Inc., Mountain View, CA (US)

(72) Inventors: Katherine Miyashiro, San Francisco, CA (US); Emily Schoenhoff, Sunnyvale, CA (US); Austin Haynes, Mountain View, CA (US); Nrupen Zaver, Mountain View, CA (US); Paul Andreotti, Santa Rosa, CA (US); Bernard Andreas, Mountain View, CA (US); Jesus Magaña, Mountain View, CA (US)

(21) Appl. No.: 18/491,749

(22) Filed: Oct. 19, 2023

Related U.S. Application Data
(60) Provisional application No. 63/417,964, filed on Oct. 20, 2022.

(51) Int. Cl. A61B 18/02

(52) U.S. Cl. CPC A61B 18/02; A61B 2018/00577

(57) ABSTRACT

Cryotherapeutic devices for targeted lunch neuromodulation therapies and associated systems and methods are disclosed herein. A cryotherapeutic device configured in accordance with embodiments of the present technology can include, for example, an elongated shaft having a distal portion and a cooling assembly positioned at the distal portions. The shaft is configured to deliver the distal portion at a target site within a bronchus of a patient. The cooling assembly includes an expandable member, an injection lumen configured to dispel a refrigerant into the expandable member, and an exhaust lumen configured to receive expanded refrigerant. The cooling assembly is configured to impart cryogenic cooling to ablate neural fibers proximate to the target site. The device can be sized to fit within a working channel of a bronchoscope such that at least a portion of the cooling assembly can be visualized during a cryotherapeutic procedure.

### CROSS-REFERENCE TO RELATED APPLICATION

This application claims the benefit of U.S. Provisional Patent Application No. 63/417,964, titled “MONITORING SYSTEMS AND METHODS FOR TARGETED LUNCH DENERVATION THERAPIES,” and filed Oct. 20, 2022, which is incorporated herein by reference in its entirety.

### TECHNICAL FIELD

The present technology relates generally to targeted neuromodulation in the lung. Several embodiments are directed to cryotherapeutic devices, systems, and methods for targeted lung neuromodulation.

### BACKGROUND

Pulmonary diseases are characterized as pathologies that adversely affect performance of the lungs, making it difficult for air-breathing animals (e.g., humans) to perform the gas exchange necessary for survival. Certain pulmonary diseases, such as chronic obstructive pulmonary disease (“COPD”), asthma, and bronchiectasis, specifically affect the airways that carry oxygen into the body. For example, COPD and asthma are characterized by a narrowing or blockage of airways which can be the result of the thickening of airway walls, alterations in the structures within or around the airway walls, or combinations thereof. For example, blockages of airway lumens can be caused by excessive intraluminal mucus and/or edema fluid. Airway wall thickening can result from inflammation, contraction or hypertrophy of the airway smooth muscle, mucous gland hypertrophy, and/or edema. Structural changes around the airway can reduce radial traction on the airway wall and lead to a narrowing of the airway. This narrowing or obstruction of airways can significantly decrease the amount of gas exchanged in the lungs resulting in breathlessness.

COPD is characterized by airflow limitation and long-term respiratory symptoms, such as shortness of breath and cough. The two most common types of COPD are emphysema and chronic bronchitis. Emphysema is defined as enlarged airspaces (alveoli) with broken down walls resulting in permanent damage to the lung tissue. Chronic bronchitis is defined as a productive cough (i.e., producing mucus or phlegm) for at least three months for two years.

### BRIEF DESCRIPTION OF THE DRAWINGS

FIG. 1A is a partially schematic illustration of a cryotherapeutic system configured in accordance with embodiments of the technology.

FIG. 1B is an enlarged side view of a cryotherapeutic device of the cryotherapeutic system of FIG. 1A.

FIG. 1C is an enlarged, partial cutaway view of a distal portion of the cryotherapeutic device of FIG. 1B configured in accordance with embodiments of the present technology.

FIG. 2 is an enlarged view of a portion of an injection tube for a cryotherapeutic device configured in accordance with embodiments of the present technology.

FIG. 3 illustrates treatment locations for cryotherapeutic procedures in accordance with some embodiments of the technology.

FIG. 4 illustrates end-view images of a cooling assembly of a cryotherapeutic device in the airway during various stages of a cryotherapeutic procedure in accordance with some embodiments of the technology.

FIG. 5 is a block diagram of a processing device configured to perform control of a cryotherapeutic system in accordance with some embodiments of the technology.

FIG. 6A is a side views of a distal portion of cryotherapeutic device configured in accordance with some embodiments of the technology.

FIG. 6B is a side views of a distal portion of cryotherapeutic device configured in accordance with some embodiments of the technology.

FIG. 7 is a flowchart of a method of cryogenically modulating nerves in accordance with some embodiments of the technology.

FIG. 8 illustrates monitoring components positioned in vasculature surrounding a target site during a cryotherapy procedure in accordance with embodiments of the present technology.

### DETAILED DESCRIPTION

Cryotherapeutic systems and methods for targeted lung neuromodulation are disclosed herein. In some embodiments, for example, a cryotherapeutic system can include a cooling assembly (also referred to as an “end effector”) at a distal portion of a shaft for performing a cryotherapy procedure at one or more target sites within airways of a lung to modulate nerves (e.g., ablate, dennervate) for the treatment of COPD and/or other pulmonary diseases. In various embodiments, the cooling assembly can include an expandable member that receives refrigerant in a manner suitable for delivering cryotherapeutic cooling to the target site. The cooling assembly can be operably coupled to a control assembly that sends and receives signals to control the operation of the refrigerant to the cooling assembly. In some embodiments, the delivery and/or exhaust of refrigerant from the cooling assembly is based on one or more intraprocedural signals associated with feedback from the cryotherapy system. The intraprocedural signal(s) can provide feedback on various parameters at or surrounding the target site (also referred to as a treatment location) such that the cryotherapy can be performed in response to the provided parameters. In some embodiments, the cryotherapeutic device may be configured for use with a bronchoscope in a cryotherapy procedure such that a user (e.g., a physician performing the procedure) may visualize the target site before, during, and/or after the procedure.

Existing treatments of a pulmonary disorder include use of systemic or inhaled medication, e.g., corticosteroids, antibiotics, bronchodilators, aerosol delivery of a “mucolytic” agent (e.g., water, hypertonic saline solution). Many of these medications have serious side effects. A thermal ablation treatment, such as bronchial thermoplasty, another treatment option, involves destroying smooth muscle tone by ablating the airway wall in bronchial branches within a lung, thereby eliminating both smooth muscles and nerves in the airway walls of the lung. The treated airways may be unable to respond to inhaled irritants, systemic hormones, and both local and central nervous system input. Accordingly, this destruction of smooth muscle tone and nerves in the airway wall may therefore adversely affect lung performance. The present technology has been shown to cryogenically ablate neural fibers for the treatment of COPD and/or other respiratory diseases, while preserving smooth muscle tone and avoiding the systemic effects of medications.

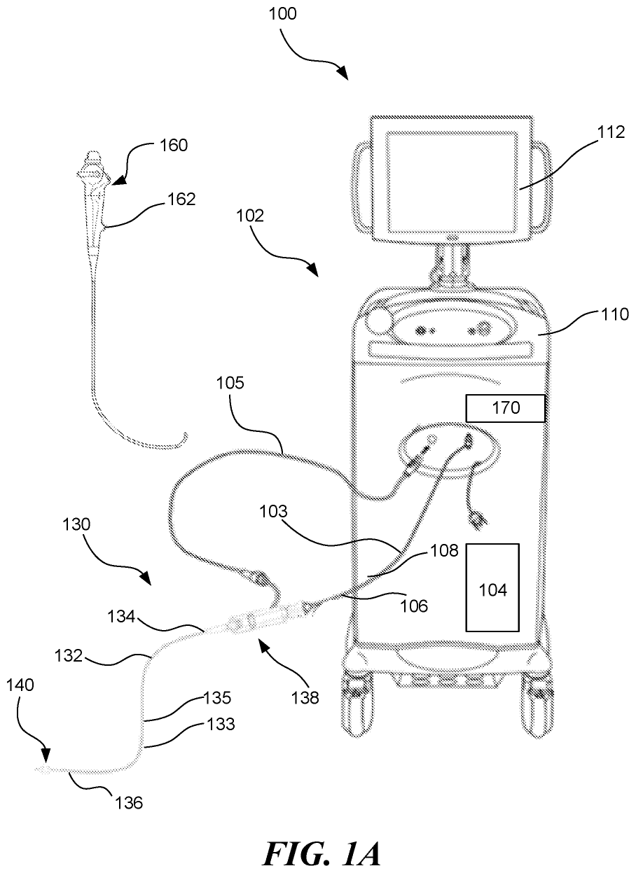

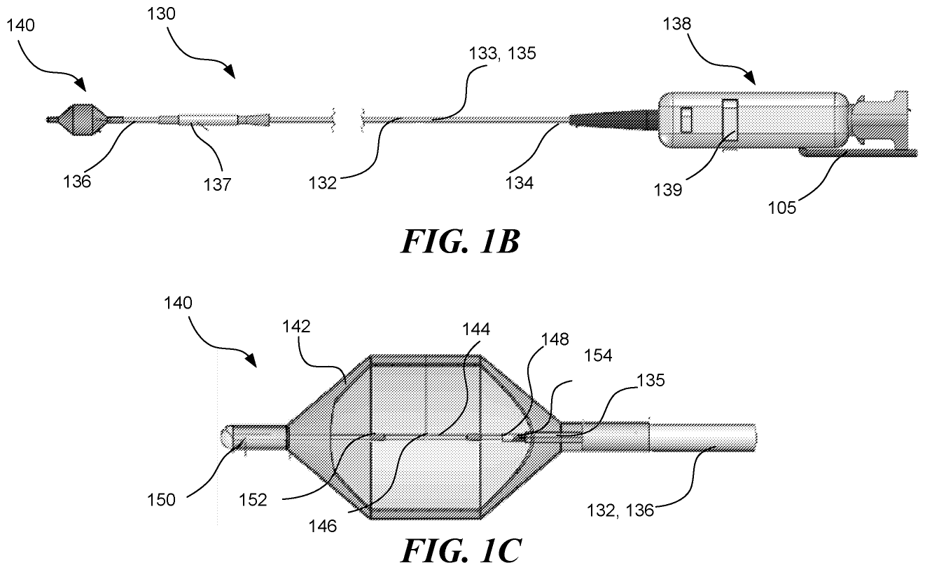

Specific details of several embodiments of the technology are described below with reference to FIGS. 1A-8. Although many of the embodiments are described below with respect to implant devices, systems, and methods for treatment and monitoring during cryotherapy for the treatment of COPD, other applications, and other embodiments in addition to those described herein are within the scope of the technology. For example, the present technology may be used for the treatment of different indications (e.g., asthma) and/or different target sites within a patient's body other than the airways, such as other portions of the respiratory system and/or other portions of the body. Additionally, several other embodiments of the technology can have different configurations, components, or procedures than those described herein, and that features of the embodiments shown can be combined with one another. A person of ordinary skill in the art, therefore, will accordingly understand that the technology can have other embodiments with additional elements, or the technology can have other embodiments without several of the features shown and described below with reference to the Figures.

With regard to the terms “distal” and “proximal” within this description, unless otherwise specified, the terms can reference a relative position of the portions of a cryotherapeutic device and/or an associated delivery device with reference to an operator and/or a location in the bronchus. For example, proximal can refer to a position closer to the operator of the device or an associated access point (e.g., the mouth), and distal can refer to a position that is more distant from the operator of the device or further from the access point.

### Bronchial Neuromodulation

Bronchial neuromodulation is the partial or complete incapacitation or other effective disruption of nerves innervating portions of the bronchi. In particular, bronchial neuromodulation includes inhibiting, reducing, and/or blocking neural communication along neural fibers (i.e., efferent and/or afferent nerve fibers) innervating the select bronchi. Such incapacitation can be long-term (e.g., permanent or for periods of months, years, or decades) or short-term (e.g., for periods of minutes, hours, days, or weeks). Bronchial neuromodulation is expected to efficaciously treat several clinical conditions characterized by increased activity of airway smooth muscle and mucus producing glands within the airways, such as COPD. The neuromodulation is expected to relax the airway smooth muscle, decrease mucus production, and/or decrease nervous system mediated inflammation and edema, which results in the reduction of airway obstruction and makes it easier to breathe.

As disclosed herein, cryotherapy can be used to partially or completely incapacitate neural pathways, such as those innervating the bronchi. Cryotherapy includes cooling tissue at a target site in a manner that modulates neural function. The mechanisms of cryotherapeutic tissue damage include, for example, direct cell injury (e.g., necrosis) and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Several embodiments of the present technology include cooling a structure at or near an inner surface of a wall of an airway such that proximate (e.g., adjacent) tissue is effectively cooled to a depth where nerves innervating the lungs reside (e.g., the phrenic nerves). For example, the cooling structure is cooled to the extent that it causes therapeutically effective, cryogenic neural modulation. Sufficiently cooling at least a portion of a nerve is expected to slow or potentially block conduction of neural signals to produce a prolonged or permanent reduction in neural activity.

Cryotherapy has certain characteristics that can be beneficial for intra airway neuromodulation. For example, rapidly cooling tissue provides an analgesic effect such that cryotherapies may be less painful than ablating tissue at high temperatures. Cryo therapies may thus require less analgesic medication to maintain patient comfort during a procedure compared to heat ablation procedures. Additionally, reducing pain mitigates patient movement and thereby increases operator success and reduces procedural complications. Cryotherapy also typically does not cause significant collagen tightening, and thus cryotherapy is not typically associated with airway stenosis.

Cryotherapies generally operate at temperatures that cause cryotherapeutic applicators to adhere to tissue. This can be beneficial because it promotes stable, consistent, and continued contact with the tissue along the inner wall of the bronchi during treatment. The typical conditions of treatment can make this an attractive feature because, for example, respiration causes the bronchi to rise and fall and thereby moves the target site, a patient can move during treatment, and/or a catheter associated with the cryotherapeutic applicator can move. Further, in procedures where a patient is asked to hold his/her breath to keep the target site stationary, the patient can exhale once the applicator is adhered to the target site. This reduces the duration of breath holding, which can often be difficult for those patients suffering from COPD or other pulmonary diseases the procedure is used to treat. Adhesion associated with cryotherapeutic cooling also can be advantageous when treating short bronchi in which stable intraluminal positioning can be more difficult to achieve.

### Select Embodiments of Cryotherapy Systems and Methods

FIG. 1A is a partially schematic illustration of a cryotherapy system 100 (also referred to as a “cryotherapeutic system 100” or “system 100”) configured in accordance with embodiments of the technology. FIG. 1B is an enlarged side view of a cryotherapeutic device 130 of the system 100 of FIG. 1A, and FIG. 1C is an enlarged, partial cutaway view of the cryotherapeutic device 130 of FIGS. 1A and 1B. Referring to FIG. 1A, the system 100 can include a console 102 operably coupled to the cryotherapeutic device 130. The console 102 can include supply component 104 (shown schematically; also referred to as a “refrigerant source”) that retains a refrigerant, such as liquid nitrogen (N₂O), carbon dioxide, and/or a hydrofluorocarbon (“HFC”; e.g., Freon®, R-410A, etc.), at a desired pressure. The supply component 104 can be in fluid communication with the cryotherapeutic device 130 via a supply line 106 (e.g., a lumen, tube) to transport the refrigerant to the cryotherapeutic device 130. An exhaust line 108 (e.g., a lumen, tube) can also be coupled to the cryotherapeutic device 130 to receive the evaporated refrigerant (e.g., nitrogen gas) after the refrigerant expands during a cryotherapeutic procedure. In some embodiments, the exhaust line 108 is coupled to a pump (not shown; e.g., a vacuum pump) and/or a control valve at the console 102 to draw the evaporated refrigerant from the cryotherapeutic device 130. This reduces the backpressure of evaporated refrigerant and, in conjunction with the supply flow rate, can increase the refrigeration power.

Referring to FIGS. 1A and 1B together, the cryotherapeutic device 130 includes a shaft 132 having a proximal portion 134 and a distal portion 136, a handle 138 at the proximal portion 134, and a cooling assembly 140 at the distal portion 136 of the shaft 132. The supply line 106 can be in fluid communication with a delivery lumen 133 extending through the shaft 132 to deliver refrigerant to the cooling assembly 140, and the exhaust line 108 can be in fluid communication with an exhaust lumen 135 extending through or alongside the shaft 132 to receive refrigerant from the cooling assembly 140. In some embodiments, the supply line 106 and the exhaust line 108 define the delivery lumen 133 and exhaust lumen 135. The shaft 132 is configured to locate the distal portion 136 at a treatment site in the airways proximate (e.g., in or near) a desired site within the bronchus, and the cooling assembly 140 is configured to deliver a therapeutically effective cryogenic nerve modulation to the target site. The shaft 132 can apply cryogenic cooling (e.g., at temperatures of −100° C. to −255° C., −90° C., or other suitable temperatures) for therapeutically effective neuromodulation at one or more target sites within the bronchus is expected to relieve the symptoms of and/or treat various respiratory diseases, such as COPD.

In some embodiments, the cryotherapeutic device 130 is configured to be used with a bronchoscope 160 that can be used to visualize the target site and aid in delivery of the cooling assembly 140 to the target site within the bronchus. For example, the distal portion of the bronchoscope 160 can be inserted into the nose or mouth of a subject (e.g., a human patient), and the distal portion 136 of the shaft 132 can be threaded through a working channel 162 of the bronchoscope 160 to navigate the cooling assembly 140 to a position at or near the target site. The cooling assembly 140 can then be exposed from the bronchoscope 160 (e.g., by moving the shaft 132 distal relative to a distal opening of the working channel 162 or retracting the bronchoscope 160, and the cooling assembly 140 can then deliver cryotherapeutic cooling to tissue at the target site. In such embodiments, the bronchoscope 160 can provide direct visualization to guide the cooling assembly 140 to one or more desired locations within the airways, as well as provide visualization during and/or after cryotherapeutic cooling. Suitable bronchoscopes 160 can include, but are not limited to, those manufactured by Olympus Corporation of the America of Center Valley, Pennsylvania.

The supply component 104 can include a single-use refrigerant cartridge for storing refrigerant under pressure and/or a larger container (e.g., a tank, refillable cylinder, or the like) that retains a sufficient volume of refrigerant to perform multiple cryotherapy procedures. The refrigerant housed within the supply component 104 may be liquid nitrogen, carbon dioxide, a hydrofluorocarb on (“HFC”; e.g., Freon®, R-410A, etc.), and/or other suitable compressed or condensed refrigerants that can be retained in the supply component 104 at a sufficiently high pressure to maintain the refrigerant in at least a substantially liquid state at ambient temperatures. the supply component 104 is configured to retain the refrigerant at a desired pressure. For example, in one embodiment, liquid N2O is contained in the supply container 104 at a pressure of 750 psi or greater so it is in at least a substantially liquid state at ambient temperatures. In other embodiments, the refrigerant can include. For example, when the refrigerant is liquid N₂O, the supply component 104 can retain the refrigerant at a pressure of 750 psi (5,171.07 kPa) or greater to maintain the refrigerant in at least a substantially liquid state at ambient temperatures. In some embodiments, the supply component 104 can include one or more chambers (e.g., tanks, vessels) that receive the refrigerant exhaust (e.g., nitrogen gas) produced during cryotherapy. The supply component 104 can include features, such as one or more valves, configured to control the flow of the refrigerant from the supply component 104 to cooling assembly 140 at the distal portion 136 of the cryotherapeutic device 130. A flow sensor may be included in or coupled to the supply component 104, the supply line 106, and/or the exhaust line 108 for sensing or measuring refrigerant flow to and/or from the cryotherapeutic device 130. As shown in FIG. 1A, the supply component 104 is housed within or otherwise carried by the console 102 and coupled to the shaft 132 of the cryotherapeutic device 130 via an umbilical 103. In some embodiments, the supply component 104 can be separate from the console 102.

As further shown in FIG. 1A, the system 100 can further include a control component 110, such as a computing device, that receives information, data, signals, and/or other feedback from various components of the system 100 and, in some embodiments, provides the detected information to an operator before, during, and/or after a cryotherapeutic procedure. For example, the control component 110 can include a display 112 that provides information related to the rate of refrigerant delivery, rate of exhaust, temperature, imaging (e.g., from the bronchoscope 160), location information, patient vitals, timers, and/or other information associated with the cryotherapy procedure and/or the patient. The display 112 can be a monitor, touch screen, LCD display, and/or other type of device that communicates information to those associated with the procedure. In some embodiments, the control component 110 is configured to communicate the information associated with the procedure to one or more remote devices (e.g., a laptop, tablet, database) in the procedure room and/or at a remote location (e.g., via Bluetooth, Wi-Fi, ethernet, and the like).

In various embodiments, the control component 110 can also be configured to generate control signals that control the delivery of refrigerant from the supply component 104, control the operation of the cryotherapeutic device 130, and/or send/receive signals from the cryotherapeutic device 130 and/or other devices associated with the cryotherapeutic procedure (e.g., patient vitals, the bronchoscope 160, sensors) to control other components of the system 100. For example, during a cryotherapy procedure, the control component 110 may control the operation of the cryotherapeutic device 130 to initiate, maintain, adjust, pause, stop, and/or resume refrigerant delivery to a treatment location of the subject in response to user inputs and/or an intraprocedural signal (e.g., detected from a sensor associated with the system 100).

In the embodiment illustrated in FIG. 1A, the console 102 is a singular unit that includes the control component 110 and the supply component 104. For example, the console 102 can include a cart (e.g., including wheels) that carries the control component 110, the supply component 104, and/or other components of the system 100, such that the components can be transported together to suitable locations between and/or during cryotherapy procedures (e.g., different procedure rooms, different locations within a procedure room). In other embodiments, components of the console 102 can be separated from each other. For example, the supply component 104, the control component 110, and/or the display 112 can be operably connected but separated from each other. In some embodiments, the bronchoscope 160 is operably coupled to a separate console and/or separate display from the console 102 such that imaging from the bronchoscope 160 is provided on a separate display.

Referring now to FIGS. 1B and 1C, the cooling assembly can include an expandable member 142, such as a balloon, attached to the distal end portion 136 of the elongated shaft 132 and an injection tube 144 extending from the shaft 132 into the expandable member 142. The injection tube 144 can be in fluid communication with the supply line 106 (FIG. 1A) and the refrigerant supply component 104 (FIG. 1A), via the delivery lumen 133, to deliver refrigerant to the chamber defined by the expandable member 142. For example, the injection tube 144 can include one or more openings 146 selectively positioned along the injection tube 144 to expel refrigerant to desired locations within the expandable member 142 and/or select portions thereof. In some embodiments the refrigerant can be delivered at a pressure between 200 psi to 760 psi, and/or other suitable pressures. The interior chamber of the cooling assembly 140 can also be in fluid communication with a refrigerant exhaust lumen port 148 at a distal end portion of the exhaust lumen 135 to remove refrigerant from the expandable member 142. In the illustrated embodiment, for example, the exhaust lumen 135 is sized larger than the injection tube 144 and extends around a proximal portion of the injection tube 144 such that the gap between the injection tube 144 and the interior wall of the exhaust lumen 135 defines the exhaust port 148. The injection tube 144 and the exhaust lumen 135 and associated port 148 may be sized or configured relative to each other based on one or more factors including a desired flow rate and/or pressure of fresh refrigerant to a target site, a desired flow rate and/or pressure of refrigerant exhaust removed from the target site, the extent of expansion of the fresh refrigerant at the target site, combinations thereof, and/or other suitable factors. In some embodiments, for example, the exhaust shaft can have an outer diameter of 2.3 mm. In some embodiments, the exhaust lumen 135 can exhaust refrigerant at a high rate (e.g., 3,000-6,000 sccm) to quickly cycle the refrigerant through the expandable member 142. In other embodiments, the exhaust shaft dimensions and/or the exhaust rate may differ.

The elongated shaft 132, the delivery lumen 133, and/or the exhaust lumen 135 may be made of a material that is substantially inert to the refrigerant, substantially insert to and biocompatible with the physiological environment of the airway, and/or sufficiently flexible at or near the temperature of refrigerant exhaust. In some embodiment, for example, the elongated shaft 132 may be made of a multilayer braided thermoplastic elastomer material (e.g., thermoplastic polyurethanes, thermoplastic copolyester (e.g., PEBAX)) and can have an outer diameter of 0.09 inch (or 2.3 millimeters). The delivery lumen 133 and/or the injection tube 144 can be made of a material that is substantially inert to the refrigerant, substantially inert to and biocompatible with the physiological environment of the airway, and/or sufficiently flexible at or near the temperature of fresh refrigerant or refrigerant exhaust to be delivered to a target site in the airway while sustaining the pressure of the fresh refrigerant or the refrigerant exhaust and the pressure and/or temperature drop when fresh refrigerant is sprayed. For example, the injection tube 144 can be made of Nitinol. In various embodiments, the shaft 132 and/or the injection tube 144 can be made from other suitable materials and/or have different dimensions suitable for delivery to the target site.

The expandable member 142 can have two configurations: a delivery state and an expanded state (shown in FIGS. 1B and 1C). In the delivery state, the expandable member 142 may have a low profile (e.g., be deflated) to facilitate its movement through the airways before and/or after cryotherapeutic cooling, e.g., when extending the cooling assembly 140 beyond the distal end of the bronchoscope 160 (FIG. 1A) to be in position for cryotherapeutic cooling (e.g., via delivery of pressurized liquid nitrogen with a low temperature and a high pressure) to or near a target site, and/or when retracting the expandable member 142 and the injection tube 144 back into the distal end of the bronchoscope 160 for removing the cooling assembly 140 from the target site after the treatment is completed. Under the deflated configuration, the expandable member 142 may be at least partially retracted in the elongated shaft 132 (or the bronchoscope 180).

In the expanded state, the expandable member 142 is inflated (e.g., via a lumen in communication with the interior of the expandable member 142) or otherwise expanded to define an interior chamber suitable for receiving refrigerant. For example, the expandable member 142 can be positioned beyond the distal end of the bronchoscope 160 such that it is positioned at the target site and expanded to abut and/or press against adjacent tissue of the airways. When the fresh refrigerant is being delivered from the injection tube 144 into the expandable member 142 via, e.g., one or more openings 146 on the injection tube 144, the refrigerant undergoes rapid expansion due to the pressure drop, thereby transitioning from liquid to gas, and effectuating the rapid cooling of the surrounding tissue for cryotherapeutic neuromodulation. Refrigerant continues to be delivered to within the expandable member 142, and thus the already expanded refrigerant exhausts through the exhaust port 148 to be guided away from the target site via the exhaust lumen 135.

In the inflated configuration, the expandable member 142 can expand radially to conform to the shape of the airway so that the refrigerant can touch or in close proximity to the target site on the wall of the airway.

The expandable member 142 can be sized and shaped such that, when in the expanded state, the expandable member 142 can oppose the inner walls of the adjacent airway section to secure the expandable member 142 in place. For example, the expandable member 142 may be configured to appose the internal wall of the airway circumferentially at a cross section. Further, the expandable member 142 may be made from a compliant and/or semi-compliant material that allows the expandable member to fit within various different airway sizes while also conforming at least partially to an interior wall of an airway so as to press against or abut the tissue. For example, the expandable member 142 can be sized to have a maximum outer diameter of 15 mm-10 mm and/or other diameters therebetween. In some embodiments, the expandable member 142 can be used to adequately press against and treat airway lumens have a diameter of 12-15 mm, which is the typical size of the left lower lobe bronchus and the right intermediate lobe bronchus, and/or lumens as small as 9 mm (e.g., the typical size of the right upper lobe bronchial). In some embodiments, the expandable member can treat smaller or larger airway lumens. The expandable member 142 can also be made from a material that provides for and can withstand rapid heat transfer to allow for the cryotherapeutic cooling of adjacent and surrounding tissue. The material may be substantially inert to and biocompatible with the physiological environment of the airway, and/or sustain the temperature of the refrigerant expelled from the injection tube 144 at which cryotherapy proceeds. For example, the expandable member 142 may be made of polyurethane, polyethylene, polytetrafluoroethylene (PTFE), polyvinyl chloride (PVC), nylon, PEBAX, silicone, polyimide, polypropylene, or the like, or a combination thereof.

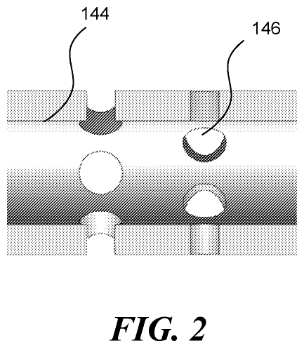

The one or more openings 146 along the injection tube 144 can be located in select positions along the length and/or circumference of the injection tube 144 to provide for a desired cryoablation pattern. For example, the openings 146 can be oriented in a manner that creates one or more fully circumferential lesions that are 360° at a cross section. This cryoablation pattern may be further enhanced by delivering the refrigerant from the injection tube 144 substantially uniformly in the circumferential direction along a desired cross section. This may be achieved by, for example, arranging openings 146 on the injection tube 144 substantially uniformly in the circumferential direction. FIG. 2 provides an example of openings 146 arranged in a pattern along the injection tube 144. In some embodiments, the openings 146 are oriented in a manner that provides cryoablation along a non-fully circumferential cross section (e.g., a hemispherical portion of the airways, spaced apart zones along a length or radial portion of the airways).

As another example, when denervation via cryoablation involves creating one or more lesions that are less than 360° at any cross section, the expandable member 142 may exhibit a non-occlusive cross section such that the cross-section of the expandable member 142 touches only a portion of a circumferential segment of the internal wall at any cross section of the airway. For example, the expandable member 142 may have non-occlusive and eccentric cross sections configured to form ablation of a linear shape, a curved shape, or a spiral shape, over a segment of the airway. The non-occlusive expandable member 142 may allow lung venting, clearing mucus, or positioning a device (e.g., a guidewire, a mucus clearing device), or the like, or a combination thereof, when the expandable member 142 is at the inflated configuration.

As further shown in FIG. 1C, the cooling assembly 140 can include an atraumatic tip portion 150 that is rounded or otherwise shaped to provide an atraumatic leading surface of the cooling assembly 140. The tip portion 150 can also or alternatively provide a support structure to which a distal end portion of the injection tube 144 and/or the distal end portion of the expandable member 142 are supported or otherwise secured.

In some embodiments, the cooling assembly 140 can include one or more sensors 154 that detects one or more parameters within the cooling assembly 140. For example, the sensor 154 can be a thermocouple that measures the temperature within the expandable member 142. This information can be communicated to the console 102 (FIG. 1A) such that the operator can track the temperature and confirm the desired cooling temperatures are met. In some embodiments, the sensor 154 may measure pressure within the expandable member 142, the flow rate of the exhaust, flow rate of the injection tube 144, and/or other parameters.

In various embodiments, the cooling assembly 140 can further include a valve, e.g., a needle valve, a Joule-Thomson throttle, a throttle element, or the like, for providing a pressure drop and a lower downstream temperature, thereby alter and/or enhance the effect and/or efficiency of the cryotherapeutic ablation. For example, a Joule-Thomson throttle can recover work energy from the expansion of the refrigerant resulting in a lower downstream temperature.

As further shown in FIG. 1C, the cooling assembly 140 can include one or more radiopaque markers 152 positioned along the injection tube 144 to allow for location guidance. In other embodiments, the radiopaque markers 152 can be positioned elsewhere on the cooling assembly 140, such as on the expandable member 142 and/or along the shaft 132.

As shown in FIG. 1B, the shaft 132 can further include a balloon protector assembly 137 to extend over and/or adjacent to the expandable member 142 to protect the components of the cooling assembly 140 as it moves into and out of the bronchoscope 160 (FIG. 1A). The protector assembly 137 can further impart stiffness to the distal portion 136 of the shaft 132.

In some embodiments, the injection tube 144 may include an external tube and a delivery sheath where the refrigerant delivery holes 146 are located. The delivery sheath may extend beyond or retract into the injection tube 144. The number (or count) of the refrigerant delivery holes 146 operable to dispel refrigerant to the interior of the expandable member 142 may be adjusted by adjusting the length of the delivery sheath extending beyond the external tube of the injection tube 144. In some embodiments, the delivery sheath may be rotatable such that the direction at which the refrigerant is sprayed into the expandable member 142 may be adjusted by rotating the delivery sheath. The adjustment (e.g., rotation, extension, or retraction) of the delivery sheath may be controlled or implemented under the control of the handle 138 (FIG. 1B) and/or manually by a user by manipulating a portion of the cryotherapeutic device 130.

Referring back to FIG. 1A, in some embodiments, the system 100 may include or be communicatively coupled to an intraprocedural monitoring component 170. The intraprocedural monitoring component 170 be operatively coupled to the control component 110 and/or integrated therewith. The intraprocedural monitoring component 170 may acquire signals associated with the cryotherapeutic procedure before, during, and/or after a cryotherapy procedure. The intraprocedural signals can indicate a status of the target site, regions surrounding the target site, and/or parameters associated with the cryotherapy treatment. The monitoring component 170 can detect signals related to the parameters of the tissue, neural fibers, and/or other structures at or near the target site. For example, the monitoring component 170 can provide information that indicates when tissue and/or other structures are at or nearing a reparable damage to tissue, when tissue and/or other structures are nearing irreparable damage at or near the target site, a thermal profile at or near the target site, images at or near the target site, the effect of the cryotherapy on nerves at the target site, the presence of specific structures at or near the target site, and/or a propagation of a cold line at or near the target site. During cryotherapy, the refrigerant can be applied at or near the target site, to modulate neural fibers (e.g., sympathetic nerves) at or near the target site. The cryotherapy may include multiple refrigerant application cycles, with thawing during a time interval between two consecutive refrigerant applications. The damage may be reparable or irreparable, depending on one or more factors including, e.g., the temperature of the refrigerant, the flow rate of the refrigerant, the duration of the time period during which the refrigerant is applied at or near the target site, the duration of an interval between two consecutive refrigerant applications at or near the target site, or the like, or a combination thereof. In some embodiments, exemplary cooling cycles can have durations of 10 seconds to 480 seconds (e.g., 240 seconds). In other embodiments, the cycle times may differ.

In some embodiments, the intraprocedural monitoring component 170 can be configured to transmit the intraprocedural signals to the control component 110 before, during, and/or after a cryotherapy procedure such that the control component 110 may control the refrigerant delivery based on the intraprocedural signals. The intraprocedural monitoring component 170 can transmit information based on the detected to a user (e.g., displayed on the display 112, displayed on a mobile device of the user), and the user may provide a user instruction to the control component 110 such that the control component 110 adjusts the procedure based on the user instruction. In some embodiments, these controls can be automated based on the feedback signals.

In some embodiments, the intraprocedural signals detected by the monitoring component 170 can include electrical signals, imaging data, physiological parameters, and/or an operation parameter of a support device (e.g., the operation of a ventilation device) that is coupled to the object during cryotherapy. The information can be provided in real time and/or provided as a change between different points in time.

The intraprocedural monitoring component 170 can be operable coupled to a monitoring device along the shaft 132, the handle 138, and/or the cooling assembly 140 to measure various parameters of the surround anatomy and the effects of the cryotherapy. The monitoring device can include, for example, a temperature sensor (e.g., a thermocouple sensor), an oxygen meter, a CO₂ meter, a pressure sensor, an electrode, and/or an imaging device. In some embodiments, the monitoring device can be a device separate from the cryotherapeutic device 130 and delivered separately at or near the target site. In some embodiments, the monitoring component 170 can be a device external to and in communication with the control component 110, the display 112, and/or a user device (e.g., a mobile device, tablet).

Targeted lung denervation performed with the system 100 can denervate the bronchial branches of the vagal nerve to reduce hyperreactivity, thereby reducing airway constriction, inflammation, and/or mucus production. Targeted lung denervation performed according to embodiments of the technology is expected to produce a therapeutic effect in COPD patients including, e.g., reduced occurrence or severity of exacerbations, an altered course of decline in a Pulmonary Function Test (PFT), and/or improvement in the quality of life of a patient.

The system 100 is configured to affect tissue at a depth beyond the tissue which it contacts so as to affect structures, such as nerves, positioned a distance away from the tissue in contact with the cooling assembly 140. This “ablation zone,” imparts thermal injury to neural fibers a distance from the bronchial lumen. The distance from the bronchial lumen and size of the ablation zone can depend, at least in part, on the duration of ablation and the number of cycles of ablation. For example, the system 100 can ablate neural fibers positioned 1 mm-3 mm from the inner wall of a bronchial lumen. In some embodiments, the system 100 can impart thermal injury to neural fibers more than 3 mm from the inner wall of the bronchial lumen. In some embodiments, the thermal injury can be acute and in others the injury occurs a time period after the initial ablation procedure (e.g., 7-30 days after). In some embodiments, the thermal injury to certain portions of the affected region (e.g., select tissue) heal after a period of time, whereas other portions, such as neural fibers, can be completed ablated.

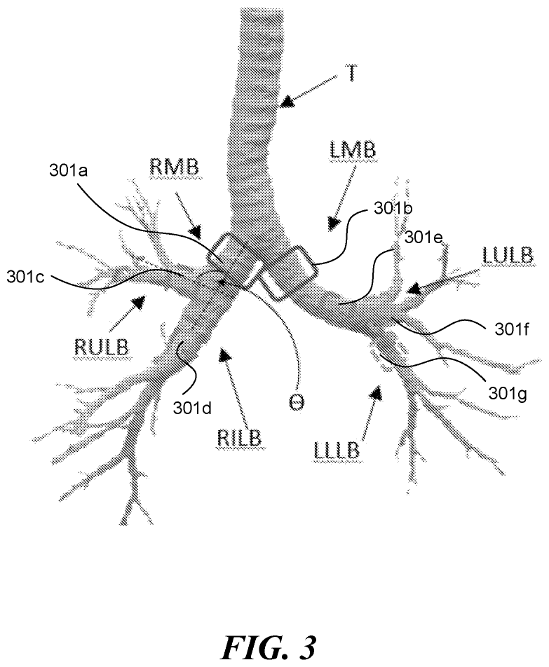

FIG. 3 illustrates airway anatomy with exemplary treatment locations for a cryotherapy procedure in accordance with some embodiments of the technology. The airway includes a trachea T that branches into a right main bronchus RMB and a left main bronchus LMB. The right main bronchus RMB further branches into smaller bronchi and bronchioles within the lung tissue in the right lung, including the right upper lobe bronchus RULB and right intermediate bronchus RILB. The left main bronchus LMB further branches into smaller bronchi and bronchioles within the lung tissue in the left lung, including the left upper lobe bronchus LULB and left lower lobar bronchus LLLB. As shown in FIG. 3, treatment sites 301 (identified individually as first through seventh treatment sites 301a-g, respectively) can be positioned within the right main bronchus RMB and the left main bronchus LMB, as well as distally therefrom beyond the bifurcations, within the right and left upper lobe bronchus RULB, LULB, the right intermediate bronchus RILB, and the lower lobar bronchus LLLB. As such, cryotherapeutic devices disclosed herein, such as the device 130 of FIG. 1A, have shafts and end effectors that are sized, shaped, and configured to navigate through tortuous airway anatomy and into airways beyond the main right and left bronchus, which have smaller diameters. For example, an angle between an axis of the right main bronchus RMB and the right upper lobe bronchus RULB, referred to the right upper lobe bronchus angle θ, can be an obtuse angle that requires a shaft of a cryotherapeutic device (e.g., the device 130 of FIGS. 1A-1C) to navigate a substantial curvature to reach any target sites within right upper lobe bronchus RULB. Accordingly, to reach a treatment location in the airway distal to the bifurcation from the right main bronchus RMB and the right upper lobe bronchus RULB, the shaft of the cryotherapeutic device 130 (FIGS. 1A-1C) must be able to flex or otherwise bend by an angle that is substantially the same as or similar to the right upper lobe bronchus angle θ. The ability to treat distal to the main bifurcation and bifurcations beyond the main right and left bronchus allows the present technology to avoid important anatomy (e.g., blood vessels, nerves not associated with the present indication) so as to avoid the cryotherapeutic cooling from adversely impacting the nearby structures.

As discussed above, the cryotherapeutic device 130 may be sized, shaped, and configured for use with a bronchoscope to navigate and visualize a cryotherapy procedure directed at a treatment location. The positioning of the cryotherapeutic device 130, or a portion thereof, may be visualized via the bronchoscope, thereby allowing the user to precisely navigate and position the catheter within the airway. This may allow for accurate targeting of a specific area in the airway that needs treatment. In addition, the bronchoscope may provide real-time visualization of the airway during the procedure, thereby allowing the user and/or the system to monitor the treatment site and make an adjustment if needed to ensure effective treatment and minimizing damage to surrounding tissues. Examples of adjustments include repositioning the cooling assembly 140, adjusting a parameter of the refrigerant delivery (e.g., the pressure and/or a flow rate of fresh refrigerant to the cryotherapeutic device 130 or refrigerant exhaust from the cryotherapeutic device 130, an injection direction of fresh refrigerant exiting the cryotherapeutic device 130 via a nozzle, refrigerant flow duration, a time interval between freezing and thawing in an ablation cycle, or the like, or a combination thereof).

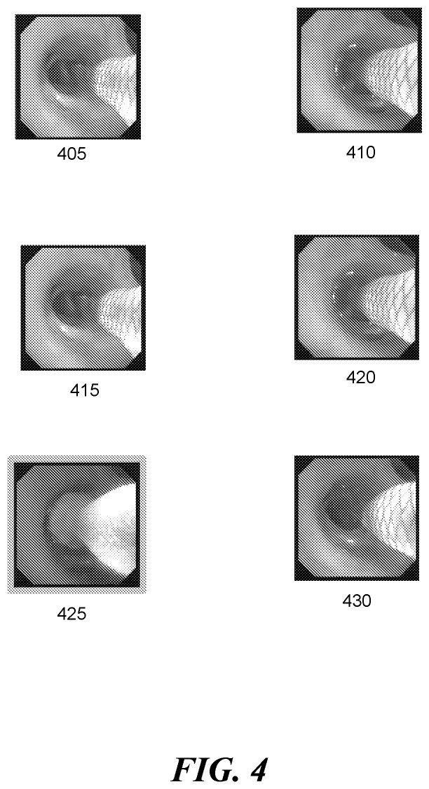

FIG. 4 illustrates bronchoscopic images of a cryotherapeutic procedure using a cryotherapeutic catheter (e.g., the cryotherapeutic device 130 of FIGS. 1A-1C) configured in accordance with embodiments of the present technology. The procedure can begin by first advancing a bronchoscope (e.g., the bronchoscope 160 of FIG. 1A) to a treatment site within the bronchus (e.g., within the right or left branch, beyond the bifurcation of the right or left branch). The distal end portion of the catheter can then be inserted through the bronchoscope's working channel and beyond the distal opening of the working channel such that it projects beyond the bronchoscope and can be visualized as shown in step 405. The expandable member can then, optionally, be inflated (e.g., with air or other gas) to confirm the exterior surface of the expandable member provides appropriate apposition against the tissue of the inner wall of the airway. Although the catheter with the semi-compliant balloon is designed to adequately appose and treat a large range of diameters, if the expandable member does not provide adequate apposition, e.g., because it is either too large or too small, the catheter can be removed, and a differently sized balloon can be used. After confirming appropriate apposition, the expandable member can be deflated and, optionally, retracted into the bronchoscope to allow the bronchoscope to capture one or more images of the treatment site.

Once the clinician is ready for the ablation, the catheter can be moved back to the target location and the position of the cooling assembly can be confirmed (e.g., via the radiopaque markers, by viewing images/video via the bronchoscope, and/or other suitable location confirmation mechanisms). The bronchoscope can optionally be positioned in a bronchoscope holder such that it remains stationary during the procedure. As shown in step 410, the expandable member can then be inflated such that it presses against the tissue at the target location and the delivery of refrigerant for cryotherapeutic neuromodulation can be initiated. In some embodiments, the patient is asked to hold his or her breath immediately before balloon inflation such that there is little to no movement at the target site, thereby maintain the position of the expandable member at the target site.

As shown in step 425, initiation of the ablation step (i.e., by delivering the refrigerant to the cooling assembly) can cause ice to form around the exterior of the expandable member. This freezes the expandable member to the surrounding tissue and stabilizes the position of the expandable member relative to the target site. In some embodiments, the cryotherapeutic cooling provided by the systems disclosed herein can cause an ice ball to form around an entire circumference of the expandable member and the abutting airway wall, or a portion thereof. With the balloon adhered or otherwise secured at the target site, the patient can release his or her breath hold. The delivery of cryotherapeutic cooling can continue (e.g., with or without breath holds) until the desired cryotherapeutic cycle of refrigerant delivery has run. Upon completion of the cryotherapeutic cooling, the procedure can continue by deflating the balloon and capturing an image of the treatment site (step 430) and/or taking measurements of the treatment site.

In some embodiments, the cryotherapeutic cooling procedure can be performed multiple times at the same target site (e.g., the balloon deflated and then reinflated for a second ablation cycle). In some embodiments, the cryotherapeutic device provide for partial circumferential ablation, and therefore the catheter can be rotated to ablate other portions along the circumference of the airways.

Combining the cryotherapeutic catheter with the bronchoscope may result in a less invasive procedure compared to traditional surgical methods, which may lead to reduced trauma to the treatment site and/or the surrounding tissues, shorter recovery times, reduced pain, and minimized risk of complications. The flexible nature of both the bronchoscope and catheter may allow access to remote or hard-to-reach areas of the airway, which is common in treating conditions in the bronchi and other branches of the airway. See, e.g., a treatment location at any one of locations as illustrated in FIG. 3.

In addition to delivering cryotherapeutic cooling, the catheter may be configured to serve one or more various other purposes including, e.g., delivery of pharmaceuticals (e.g., analgesics, local anesthesia medicines), fluids, and/or therapies directly to the target site, suction of mucus or debris, detection of data surrounding the target site, thereby allowing for tailored treatments based on the patient's condition and needs.

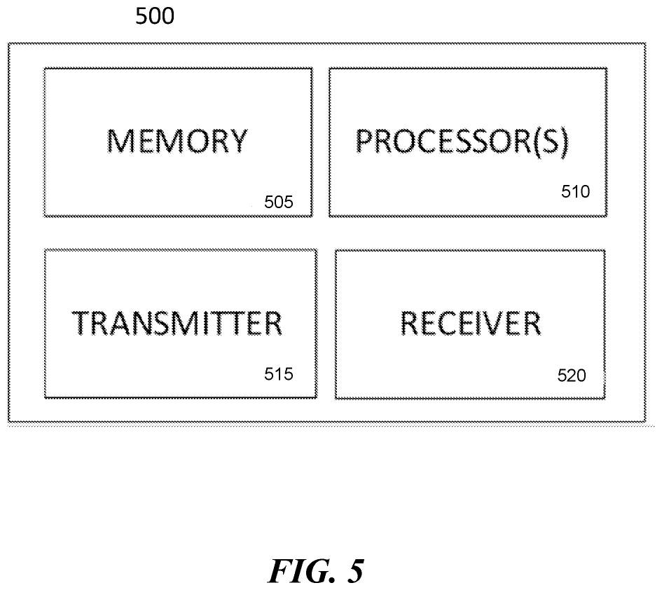

FIG. 5 illustrates a block diagram of a processing device configured to perform control of the system 100 in accordance with some embodiments of the technology. The processing device 500 may constitute an example of the control component 110 (FIG. 1A). The processing device 500 may be operably coupled to or integrated with the console 102, the display 112, input devices, the intraprocedural monitoring component 170, and/or other devices or components of the system 100 as illustrated in FIG. 1A. The processing device 500 may be configured to receives information, data, signals, and/or other feedback, and generate control signals to control the operation of the system 100 of FIG. 1A and components thereof.

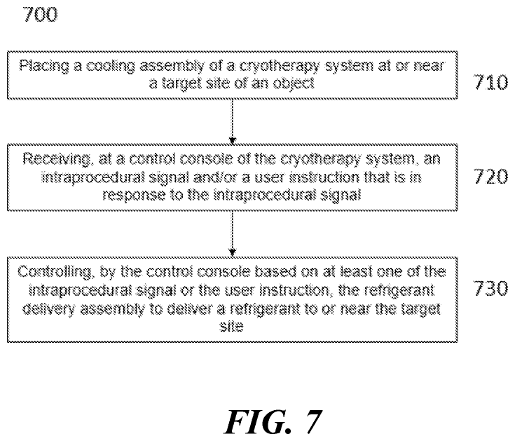

The processing device 500 may include memory 505 and processor(s) 510. The memory 505 may have instructions stored thereupon. The instructions, upon execution by the processor(s) 510, may configure the processing device 500 (e.g., the various modules or components of the processing device 500) to perform the operations described elsewhere in the present document including, e.g., those illustrated in FIG. 7. The processor(s) 510 may include at least one processor and/or control circuitry to send and receive commands, requests, and other suitable data using one or more input/output (I/O) paths.

The control circuitry may include any suitable processing, storage, and/or I/O circuitry. The processing device 500 may also include or be operably connected to a user input interface and/or user output interface. As referred to herein, a “user interface” may include a human-computer interaction and communication in a device and may include display screens for use in receiving and displaying data, input devices (e.g., a keyboard, a touchscreen, a mouse), and the appearance of a desktop. For example, a user interface may include a way a user interacts with the control components and/or the intraprocedural monitoring components.

In some embodiments, the processing device 500 may include a transmitter 515 and a receiver 520 configured to send and receive information, respectively. At least one of the transmitter 515 or the receiver 520 may facilitate communication via a wired connection and/or a wireless connection between the processing device 500 and a device or information resource external to the processing device 500. For example, the processing device 500 may receive sensor data acquired by sensors of an intraprocedural monitoring component via the receiver 520. As another example, the processing device 400 may receive input from a user via the receiver 520. As a further example, the processing device 500 may transmit a notification to a user (e.g., a medical professional, a display) via the transmitter 515. In some embodiments, the transmitter 515 and the receiver 520 may be integrated into one communication device.

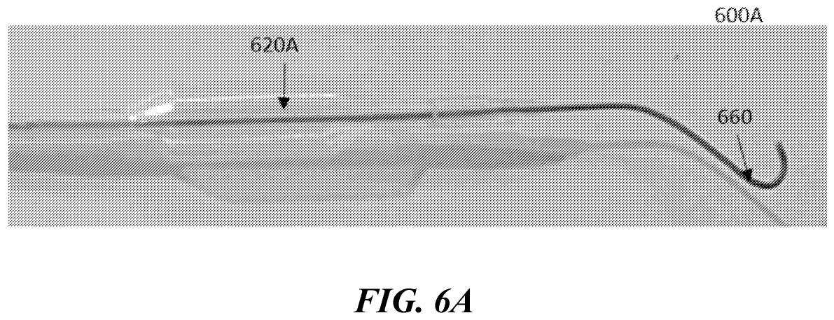

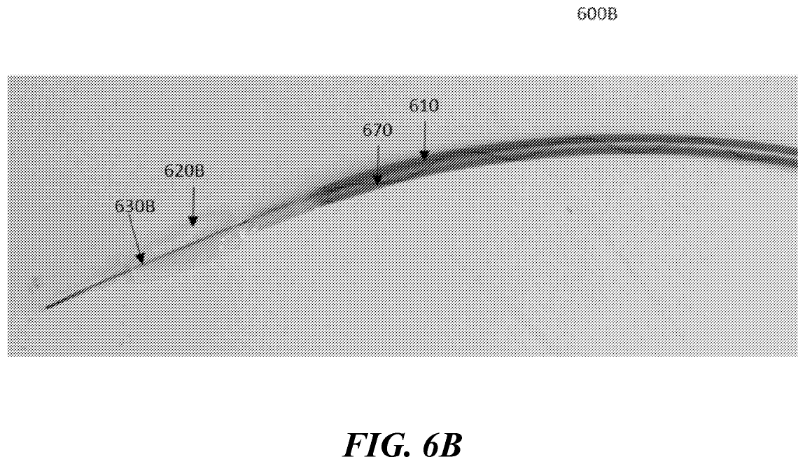

FIGS. 6A and 6B illustrate side views of various refrigerant delivery assemblies in accordance with some embodiments of the technology. The refrigerant delivery assembly 600A as illustrated in FIG. 6A includes a balloon 620A and a separate lumen 660 in fluid communication with the airway of a patient. The lumen 660 may allow lung venting, clearing mucus, or positioning a device (e.g., a guidewire, a mucus clearing device), or the like, or a combination thereof, when the refrigerant delivery assembly 600A is in place and the balloon 620A is at the inflated configuration. The refrigerant delivery assembly 600A may include an injection tube attached to an external wall of the lumen 660 that opposes the balloon 620A.

The refrigerant delivery assembly of a catheter 600B as illustrated in FIG. 6B includes a balloon 620B attached to a distal end of an elongated shaft 610 and an injection tube 630B that at least partially extend beyond the elongated shaft 610. The catheter 600B may include a delivery line 670 through which fresh refrigerant is delivered to or near a target site from a cryoconsole (e.g., console 102 as illustrated in FIG. 1). The injection tube 630B may be in fluid communication with or a distal end of the delivery line 670. The delivery line 670 may be similar to the delivery line 106 as illustrated in FIG. 1A except that the delivery line 670 is positioned on and spirals along an external wall of the elongated shaft 610 and straightened at a position close (e.g., just proximal to) where the balloon 620B is attached to the elongated shaft 610. Benefits of this exemplary configuration of the delivery line 670 may include one or more of the following: helping maintain the temperature stability of refrigerant and stabilizes ablation cycles due to the temperature stability, allow for distal flexibility; adding crush resistance, torque, and/or pushability to the catheter 600B, and/or facilitating twisting of the balloon 620B when the balloon 620B is packed for delivery in the airway (toward or away from a target site in the airway).

FIG. 7 is a flowchart illustrating a cryotherapy procedure 700 in accordance with some embodiments of the technology. In 710, a cooling assembly of a cryotherapy device of a cryotherapeutic system can be placed at or near a target site of an object (e.g., a patient). The target site may be a treatment location in the airway of the object. The treatment location may be a location distal to a bifurcation along the airway so that air passage downstream of the treatment locations can be modified. Example treatment locations may include a location at the right upper lobe bronchus, the right intermediate bronchus, the left upper lobe bronchus, the left lower lobar bronchus.

The cooling assembly may be part of a catheter sized to fit within a lumen of a bronchoscope or otherwise attached to the bronchoscope (e.g., attached to an external side of the bronchoscope) such that the refrigerant delivery assembly may be delivered to or near the target site in the airway by inserting the lumen of the bronchoscope into the airway. The positioning of the refrigerant delivery assembly can be observed or verified based on views provided by the bronchoscope and/or feedback from monitoring components of the cryotherapy system.

The cooling assembly may include an expandable member, such as a balloon, and an injection tube configured to deliver refrigerant into the balloon. The balloon may move from a first or deflated configuration to a second or inflated configuration (via delivery of a gas or other fluid into the balloon) such that the inflated balloon apposes the adjacent airway wall at the target site. Refrigerant is dispelled through openings of the injection tube into the balloon to provide cryotherapeutic cooling. The balloon may be configured to facilitate heat transfer between the tissue underneath the internal wall of the body lumen and the refrigerant in the balloon where the balloon contacts the internal wall such that at least a portion of the tissue undergoes cryoablation in the cryotherapy procedure 700.

In 720, at least one of an intraprocedural signal or a user instruction can be received at the control console of the cryotherapy system. The user instruction can be in response to the intraprocedural signal. The intraprocedural signal can be acquired while the object is being treated in the cryotherapy procedure 700 directed to the target site as described in 710. The intraprocedural signal can indicate a status of the target site, or a vicinity of the target site. The status (including a change thereof) can be caused by the cryotherapy procedure 700, and therefore, can be used to control the proceeding of the cryotherapy procedure by controlling the refrigerant delivery.

In 730, the control console can control, based on at least one of the intraprocedural signal or the user instruction, the refrigerant delivery assembly to deliver the refrigerant to or near the target site. For instance, the control console can cause the refrigerant delivery assembly to maintain the delivery of the refrigerant to or near the target site, pause the delivery of the refrigerant to or near the target site; resume the delivery of the refrigerant to or near the target site; or cause at least a portion of the refrigerant delivery assembly to move away from the target site. Merely by way of example, the control console may automatically divert or halt a refrigerant flow after a set time, using an electronic or mechanical timer to manage a valve operably connected to a cryoconsole. Alternatively, this timer may be built into the catheter, perhaps within its handle.

Some aspects of the present disclosure include a kit for use in a cryotherapy procedure in accordance with some embodiments of the technology. Components to practice the method of the present technology may be packaged and provided to medical professionals in the form of a kit. The kit may include a catheter, having a delivery line, a balloon, and an injection tube, as well as a means for connecting the catheter to a cryoconsole. Merely by way of example, the connection means may include a luer connection on the opposite end of the catheter from the injection tube. The connection means may allow a user to conveniently connect the catheter to the cryoconsole to form a sealed fluid communication between the catheter and the cryoconsole so that the catheter can receive and deliver fresh refrigerant to or near a target site in the body of a patient and guide refrigerant exhaust away from the target site. The catheter may be sized to be fit within a lumen or attached to an external side of a bronchoscope so that the catheter may be delivered to or near the target size by inserting the bronchoscope. The kit may also include user instructions advising how to use the catheter, including how to connect the catheter to a bronchoscope and/or a cryoconsole, applicable values or value ranges of one or more operational parameters (e.g., the pressure, the flow rate, the temperature of refrigerant, or the like, or a combination t hereof, that are suitable for the catheter), compatible bronchoscopes and/or cryoconsoles where the catheter may be used. The kit may be sealed in a sterile manner for opening at the stie of a procedure.

### Select Embodiments of Monitoring Systems and Methods

As discussed above, cryotherapeutic systems disclosed herein can be configured to detect signals (referred to as “intraprocedural signals) from various devices (e.g., sensors) associated with the systems disclosed herein. These signals can include electrical signals, imaging data, physiological parameters, and/or an operation parameter of a support device (e.g., the operation of a ventilation device) that is coupled to the object during cryotherapy. The information can be provided in real time and/or provided as a change between different point in time.

The intraprocedural monitoring system (e.g., the monitoring component 170 of FIG. 1A) can include one or more monitoring components carried by a cryotherapeutic device, a bronchoscope, and/or other components of a cryotherapeutic system, and can be delivered into the body before, during, and/or after cryotherapy to measure various parameters of the surround anatomy and the effects of the cryotherapy. The monitoring component can include a temperature sensor (e.g., a thermocouple), an oxygen meter, a CO₂ meter, a pressure sensor, an electrode, and/or an imaging device. In some embodiments, the monitoring component can be a separate device delivered separately at or near the target site or to surrounding anatomy (e.g., surrounding blood vessels).

When the monitoring system includes a temperature sensor (e.g., a thermocouple), the temperature sensor can measure the temperature at or near the target site (e.g., in the airways, tissue of the airways, tissue of the esophagus, the surrounding blood vessels, and/or other structures) over a time period before, during, and/or after cryotherapy application. A thermal profile at or near the target site may be determined based on the measured temperature. For instance, if the correlation between different thermal profiles and corresponding cell/tissue damage in the object is known, the status (including a status change) at or near the target site of the object may be determined or estimated.

When the monitoring system includes an oxygen meter, the oxygen meter can measure the oxygen level in the air at or near the target site located in the airway. The CO₂ meter can measure the CO₂ level in the air at or near the target site located in the airway. An oxygen level and/or an CO₂ level, or a change thereof, can correlate with the metabolism at or near the target site, and therefore indicate a status (e.g., cell/tissue damage caused by the cryotherapy) at or near the target site. Therefore, on the basis of the intraprocedural signal including the temperature, the oxygen level, and/or the CO₂ level, at or near the target site over time, the control component 110 may control the refrigerant delivery by the cooling assembly 140.

When the monitoring system includes a pressure sensor, the pressure sensor may measure the pressure at one or more points at or near the target site. For instance, the pressure at each of two different points at or near the target site can be monitored over time during cryotherapy, and a pressure difference over time may be determined based on the pressure at each of the two different points over time; a change in the pressure difference over time can indicate the status of the target site, or a vicinity thereof. As another example, considering that the airway is usually in proximity with the corresponding arterials, arterial pressure can be monitoring as an approximate of the pressure at or near the target site in the airway. On the basis of the intraprocedural signal including the pressure at each of the two different points and/or the pressure difference between the two different points over time, a control component (e.g., the control component 110 of FIG. 1A) may control the delivery of refrigerant to the cryotherapeutic device.

In some embodiments, the monitoring component includes one or more electrodes (e.g., an electrode pair) for measuring electrical at or near the target site. The electrical signal can include, e.g., resistance or impedance of a portion of the object at or near the target site, indicating or relating to the electrical conductivity, or a change thereof, of the portion of the object. The change in electrical conductivity can be due to a change in cell/tissue composition and/or a change in metabolism caused by, e.g., cell/tissue damage, indicating a status (including a status change) at or near the target site. On the basis of the intraprocedural signal including the electrical signal over time, the control component may control the delivery of refrigerant to the cryotherapeutic device.

In some embodiments, the intraprocedural signals may be used to provide image data providing a representation of a cold/freeze line and/or a representation of the frozen cells/tissue (e.g., appearing as an ice ball in an image). The cold/freeze line can be the interface between the frozen cells/tissue and normal or unfrozen cells or tissue. The imaging device can include an ultrasound device, a fluoroscope, a computed tomography (CT) scanner, a tomosynthesis scanner, etc. In some embodiments, the imaging device can be portable so that it can be moved to the operation room to perform intraprocedural monitoring.

In various embodiments, monitoring assemblies as disclosed herein can be used to detect physiological parameters to select target sites, determine conditions during cryotherapy, and/or determine the effects of the cryotherapy. An anesthesia plan can be set to allow for spontaneous respiration before, during, and/or after an ablation procedure to intraoperatively monitor a physiological parameter, or a change thereof, before and after an ablation procedure. Such a physiological parameter, or a change thereof, may be used as an intraprocedural signal used to guide the proceeding of the ablation procedure. Examples of the physiological parameter, or a change thereof, to be monitored can include a change in the CO₂ level within the airway near the target site (e.g., CO₂ build up values), a change to the rate of respiration, a change in the respiratory sinus arrhythmia, a change in the metabolic activity (metabolic tracking/monitoring in which one or more metabolic parameters are tracked or monitored), the arterial pressure in the artery located in a close proximity to the target site in the airway, a cardiac plexus activity, a change in oxygenation needs from anesthesia before, after, and/or during the ablation procedure.

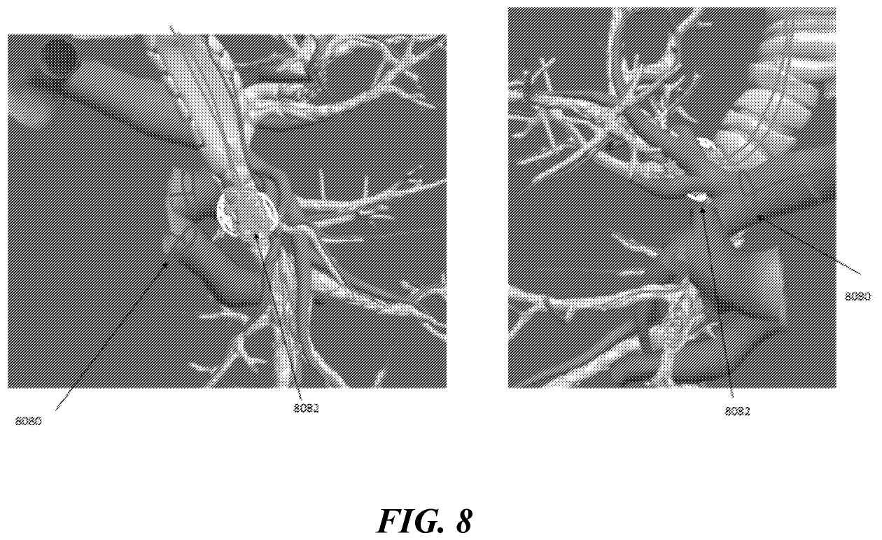

In various embodiments, monitoring assemblies as disclosed herein can employ an electromyography (EMG) catheter or another metallic frame with one or more sensors. FIG. 8 illustrates various EMG catheters with EMG sensors 8080 (e.g., a metallic frame) positioned in veins and arteries surrounding a target site within the bronchus near the location of a cryotherapeutic end effect 8082 (e.g., the cooling assembly 140 of FIGS. 1A-1C) before, during, and/or after a cryotherapy procedure. An electrical signal acquired using the EMG sensor 8080 may be used as an intraprocedural signal to guide the proceeding of an ablation procedure and/or determine efficacy. The EMG sensor 8080 can be a metallic frame configured to expand to appose the inner wall of the lumen in which it is positioned (e.g., within a vein or artery). The EMG sensor 8080 can be placed in a neighboring vein or artery that is located in a close proximity to the target site in the airway. For example, the electrical resistance in a neighboring pulmonary vein or artery may be monitored at a specified distance from the catheter, considering that metallic elements may generate a signal to echolocate with the EMG sensor 8080, or relative positioning of the cryotherapy end effector and surrounding anatomy may be confirmed on fluoroscopy. The EMG sensor 8080 can stay in place during the ablation procedure, and a modification of the EMG signal during the ablation procedure can be due to the formation and propagation of freezing, which in turn can signify that a specific ablation depth has been achieved. In various embodiments, a mapping catheter may be used in a similar neighboring location to look for electrical signals (resistance, impedance) before, during, and/or after the ablation procedure as electrical conduction properties can change due to freezing and cell injury/edema after treatment by the ablation procedure.

In various embodiments, the monitoring system can include an ECG catheter in the stomach or esophagus can be used to intraoperatively monitor an esophageal plexus or main vagus nerve activity. An electrical signal acquired using the ECG catheter may be used as an intraprocedural signal to guide the proceeding of an ablation procedure. For example, the ablation procedure can be stopped immediately if a modification to the esophageal ECG signal is identified.

In various embodiments, the monitoring system can include detection components used for phrenic nerve monitoring, and intraoperative monitoring can be performed by pacing main vagus nerve activity to highlight a pulmonary nerve activity, thereby better amplifying differences in the pulmonary nerve activity during and after an ablation procedure. An electrical signal corresponding to the pulmonary nerve activity may be used as an intraprocedural signal to guide the proceeding of the ablation procedure.

In various embodiments, the monitoring system can include components that provide for live imaging, e.g., a CT scanner, to monitor the freezing of cells and/or tissue at or near a target site of an object. Imaging data so acquired using an imaging device may be used as an intraprocedural signal to guide the proceeding of the ablation procedure. For instance, using live CT imaging, frozen cells and/or tissue can appear as an ice block (e.g., an ice ball) gradually change (e.g., growing) in the CT images so acquired.

In various embodiments, the monitoring system can include components that detect and/or receive information related to mechanical ventilation parameters. A change in mechanical ventilation needs during an ablation procedure, a change in respiration rate of an object being treated in the ablation procedure can be intraoperatively monitored in order to guide the proceeding of the ablation procedure. An operation parameter of a support device (e.g., a ventilation device), the respiration rate of the object, or the like, or a combination thereof, may be used as an intraprocedural signal to guide the proceeding of the ablation procedure.

### EXAMPLES

The following examples are illustrative of several embodiments of the present technology.
1. A catheter, comprising:
   an elongated shaft configured to house a delivery line and an exhaust lumen, wherein:
      the delivery line is configured to receive and deliver refrigerant to or near a target site in an airway, and
      the exhaust lumen is configured to guide refrigerant exhaust away from the target site,
   a balloon connected to a distal end of the elongated shaft, and
   an injection tube in fluid communication with the delivery line and configured to spray the refrigerant into the balloon through which the refrigerant is allowed thermal communication with the target site, wherein
      the catheter is sized to fit within a lumen of a bronchoscope such that at least a portion of the balloon or the injection tube is configured to be delivered to or near the target site by inserting the lumen of the bronchoscope in the airway.
2. The catheter of example 1, wherein:
   the balloon has a deflated configuration and an inflated configuration,
   the balloon is at the deflated configuration when the balloon is being delivered toward or retracted from the target site, and
   the balloon is configured to appose an internal wall of the airway at the inflated configuration.
3. The catheter of example 2, wherein the balloon is configured to partially occlude a portion of the airway where the balloon apposes the internal wall when the balloon is at the inflated configuration.
4. The catheter of example 2, wherein the balloon is configured to completely occlude a portion of the airway where the balloon apposes the internal wall when the balloon is at the inflated configuration.
5. The catheter of example 2, wherein:
   the balloon is configured to at least partially retract within the elongated shaft when the balloon is at the deflated configuration, and
   at least partially extend beyond the elongated shaft when the balloon is at the inflated configuration.
6. The catheter of any one of the preceding examples, wherein:
   the injection tube is configured to at least partially retract within the elongated shaft when the injection tube is being delivered toward or retracted from the target site, and
   the injection tube is configured to extend at least partially beyond the distal end of the elongated shaft when spraying the refrigerant into the balloon.
7. The catheter of any one of the preceding examples, wherein the injection tube has a plurality of openings through which the refrigerant is sprayed into the balloon.
8. The catheter of example 7, wherein at least one of the plurality of openings includes a nozzle or an orifice through which the refrigerant undergoes expansion and pressure drop when sprayed into the balloon.
9. The catheter of any one of the preceding examples, wherein the delivery line is located within the exhaust lumen.
10. A system for delivering refrigerant, the system comprising:
   a catheter comprising:
      an elongated shaft configured to house a delivery line and an exhaust lumen, wherein:
         the delivery line is configured to receive and deliver refrigerant to or near a target site in an airway, and
         the exhaust lumen is configured to guide refrigerant exhaust away from the target site,
      a balloon connected to a distal end of the elongated shaft, and
      an injection tube in fluid communication with the delivery line and configured to spray the refrigerant into the balloon through which the refrigerant is allowed thermal communication with the target site, wherein
         the catheter is sized to fit within a lumen of a bronchoscope such that the balloon and the injection tube are configured to be delivered to or near the target site by inserting the lumen of the bronchoscope in the airway.
11. The system of example 10, further comprising the bronchoscope configured to provide real-time visualization of at least a portion of the catheter when the catheter moves within the airway with the bronchoscope.
12. A kit for delivering refrigerant, the kit comprising the catheter of any one of examples 1-9.
13. The kit of example 12, further comprising instructions for using the catheter in a cryotherapy procedure.
14. A method for delivering refrigerant, comprising:
   placing, at or near a target site of an object, a refrigerant delivery assembly of the catheter of any one of examples 1-9;
   receiving, at a control console of a cryotherapy system, at least one of an intraprocedural signal or a user instruction that is in response to an intraprocedural signal; and
   controlling, based on at least one of the intraprocedural signal or the user instruction, the refrigerant delivery assembly to deliver the refrigerant to or near the target site.

### CONCLUSION

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present technology have been shown and described, it will be obvious to those skilled in the art that, based upon the teachings herein, changes and modifications may be made without departing from this technology and its broader aspects and, therefore, the appended claims are to encompass within their scope all such changes and modifications as are within the true spirit and scope of this technology. Furthermore, it is to be understood that the technology is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to technologies containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Conjunctive language, such as phrases of the form “at least one of A, B, and C,” or “at least one of A, B and C,” (i.e., the same phrase with or without the Oxford comma) unless specifically stated otherwise or otherwise clearly contradicted by context, is otherwise understood with the context as used in general to present that an item, term, etc., may be either A or B or C, any nonempty subset of the set of A and B and C, or any set not contradicted by context or otherwise excluded that contains at least one A, at least one B, or at least one C. For example, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}, and, if not contradicted explicitly or by context, any set having {A}, {B}, and/or {C} as a subset (e.g., sets with multiple “A”). Thus, such conjunctive language is not generally intended to imply that certain embodiments require at least one of A, at least one of B, and at least one of C each to be present. Similarly, phrases such as “at least one of A, B, or C” and “at least one of A, B or C” refer to the same as “at least one of A, B, and C” and “at least one of A, B and C” refer to any of the following sets: {A}, {B}, {C}, {A, B}, {A, C}, {B, C}, {A, B, C}, unless differing meaning is explicitly stated or clear from context.

What is claimed is:

1. A catheter, comprising:
an elongated shaft configured to house a delivery line and an exhaust lumen, wherein:
the delivery line is configured to receive and deliver refrigerant to or near a target site in an airway, and
the exhaust lumen is configured to guide refrigerant exhaust away from the target site,
a balloon connected to a distal end of the elongated shaft, and
an injection tube in fluid communication with the delivery line and configured to spray the refrigerant into the balloon through which the refrigerant is allowed thermal communication with the target site, wherein
the catheter is sized to fit within a lumen of a bronchoscope such that at least a portion of the balloon or the injection tube is configured to be delivered to or near the target site by inserting the lumen of the bronchoscope in the airway.

2. The catheter of claim 1, wherein:
the balloon has a deflated configuration and an inflated configuration,
the balloon is at the deflated configuration when the balloon is being delivered toward or retracted from the target site, and
the balloon is configured to appose an internal wall of the airway at the inflated configuration.

3. The catheter of claim 2, wherein the balloon is configured to partially occlude a portion of the airway where the balloon apposes the internal wall when the balloon is at the inflated configuration.

4. The catheter of claim 2, wherein the balloon is configured to completely occlude a portion of the airway where the balloon apposes the internal wall when the balloon is at the inflated configuration.

5. The catheter of claim 2, wherein:
the balloon is configured to at least partially retract within the elongated shaft when the balloon is at the deflated configuration, and
at least partially extend beyond the elongated shaft when the balloon is at the inflated configuration.

6. The catheter of claim 1, wherein:
the injection tube is configured to at least partially retract within the elongated shaft when the injection tube is being delivered toward or retracted from the target site, and
the injection tube is configured to extend at least partially beyond the distal end of the elongated shaft when spraying the refrigerant into the balloon.

7. The catheter of claim 1, wherein the injection tube has a plurality of openings through which the refrigerant is sprayed into the balloon.

8. The catheter of claim 7, wherein at least one of the plurality of openings includes a nozzle or an orifice through which the refrigerant undergoes expansion and pressure drop when sprayed into the balloon.

9. The catheter of claim 1, wherein the delivery line is located within the exhaust lumen.

10. A system for delivering refrigerant, the system comprising:
a catheter comprising:
an elongated shaft configured to house a delivery line and an exhaust lumen, wherein:
the delivery line is configured to receive and deliver refrigerant to or near a target site in an airway, and
the exhaust lumen is configured to guide refrigerant exhaust away from the target site,
a balloon connected to a distal end of the elongated shaft, and
an injection tube in fluid communication with the delivery line and configured to spray the refrigerant into the balloon through which the refrigerant is allowed thermal communication with the target site, wherein
the catheter is sized to fit within a lumen of a bronchoscope such that the balloon and the injection tube are configured to be delivered to or near the target site by inserting the lumen of the bronchoscope in the airway.

11. The system of claim 10, further comprising the bronchoscope configured to provide real-time visualization of at least a portion of the catheter when the catheter moves within the airway with the bronchoscope.

12. A kit for delivering refrigerant, the kit comprising the catheter of any one of claims 1-9.

13. The kit of claim 12, further comprising instructions for using the catheter in a cryotherapy procedure.

14. A method for delivering refrigerant, comprising:
placing, at or near a target site of an object, a refrigerant delivery assembly of the catheter of any one of claims 1-9;
receiving, at a control console of a cryotherapy system, at least one of an intraprocedural signal or a user instruction that is in response to an intraprocedural signal; and
controlling, based on at least one of the intraprocedural signal or the user instruction, the refrigerant delivery assembly to deliver the refrigerant to or near the target site.
